(19) United States
(12) Patent Application Publication
(10) Pub. No.: US 2013/0345454 A2
(43) Pub. Date: Dec. 26, 2013

(54) A PROCESS FOR THE PREPARATION OF PHYTOSTERYL FERULATE

(71) Applicants: Bethala Lakshmi Anu Prabhavathi DEVI, Hyderabad (IN); Bhongiri YADAGIRI, Hyderabad (IN); Sanjit KANJILAL, Hyderabad (IN); Sistla RAMAKRISHNA, Hyderabad (IN); Kuncha MADHUSUDHANA, Hyderabad (IN); Prakash Vamanrao DlWAN, Hyderabad (IN); Rachapudi Badari Narayana PRASAD, Hyderabad (IN)

(72) Inventors: Bethala Lakshmi Anu Prabhavathi DEVI, (IN); Bhongiri YADAGIRI, (IN); Sanjit KANJILAL, (IN); Sistla RAMAKRISHNA, (IN); Kuncha MADHUSUDHANA, (IN); Prakash Vamanrao DlWAN, (IN); Rachapudi Badari Narayana PRASAD, (IN)

(73) Assignee: COUNCIL OF SCIENTIFIC & INDUSTRIAL RESEARCH, NEW DELHI (IN)

(21) Appl. No.: 13/203,374

(22) PCT Filed: Feb. 25, 2010
(86) PCT No.: PCT/IN2010/000109

§ 371 Date: Jan. 9, 2012

(30) Foreign Application Priority Data
Feb. 25, 2009 (IN) 350/DEL/2009

(51) Int. Cl. C07J 9/00

(52) U.S. Cl. 552/544

(57) ABSTRACT

The present invention relates to a synthetic method for the preparation of phytosteryl ferulate using ferulic acid and phytosterols (isolated from soybean oil deodorizer distillate) comprising of (a) acetylation of ferulic acid to ferulic acid acetate; (b) esterification of ferulic acid acetate with phytosterols to obtain phytosteryl ferulate acetate; (c) deprotection of phytosteryl ferulate acetate to phytosteryl ferulate. The phytosteryl ferulate was evaluated for hypocholesteremic activity in hamsters in comparison with that of natural oryzanol isolated from rice bran oil soap stock. This study confirmed that the phytosteryl ferulate significantly lowers the elevated cholesterol levels and also interferes with the absorption of cholesterol and the effect is comparable with that of natural oryzanol and is useful as nutraceutical/food supplement. Hence phytosteryl ferulate is a potential substitute for natural oryzanol for several applications.

### FIELD OF THE INVENTION

The present invention relates to a process for the preparation of phytosteryl ferulate as nutraceutical/food supplement, an equivalent to some of the molecules present in oryzanol isolated from rice bran oil soap-stock using ferulic acid and phytosterols isolated from soybean oil deodorizer distillate. More particularly, the present invention also relates to evaluation of phytosteryl ferulate for hypocholesteremic activity in hamsters in comparison with that of natural oryzanol isolated from rice bran oil soap stock as nutraceutical/food supplement.

### BACKGROUND OF THE INVENTION

Rice bran oil is the only source for natural oryzanol. Oryzanol was first isolated from rice bran oil [Kaneko, R. and T. Tsuchiya; J. Chem. Soc. Jpn. 57,526 (1954)]; [Tsuchya, T. et al; JP 4895 (1957)] and was presumed to be a single component. Oryzanol is a mixture of esters of ferulic acid (4-hydroxy 3-methoxy cinnamic acid) with cycloartenol, 24-methylene cycloartenol, Campesterol and β-sitosterol (FIG. 1) [Rogers, E. J; Rice, S. M.; Nicolosi, R. J,; Carpenter, D. R.; McClelland, C. A.; Romanczyk, L. J., Jr. J. Am. Oil. Chem. Soc. 70, 301-307 (1993); Diack, M.; Saska, M. J. Am. Oil. Chem. Soc. 71, 1211-1217 (1994); Norton, R. A. Lipids, 30, 269-274 (1995); Xu, Z.; Godrej, J. S. J. Agric. Food Chem. 47, 2724-2728 (1999)]. Over the decades, the methods of oryzanol isolation from plant oils have been improved. Such method include isolation of cycloartenyl ferulate from plant oil using selective organic solvent for oryzanol extraction followed by chromatographic purification [Kimura, Goro Jap Pat 6314796 (1988) and Jap Pat 6314797 (1988)], isolation of oryzanols from rice bran dark oil by precipitating the sterins with aluminium sulfate followed by crystallization of oryzanols from the supernatant [Beso oils & fats Co. Ltd., Jap Pat 8295942 (1982)], highly concentrated separation of oryzanols from rice bran and rice germ oils by two-step alkali treatment [Shimuzu, Hisashi; Jap Pat 76123811 (1976)]. Extraction of oryzanols from rice bran soap stack with diethyl ether at pH 9.5 followed by its chromatographic purification on a neutral alumina column [G. S. Seetharamaiah, and J. V. Prabhakar; J. Food Science Technology, 23,270 (1986)]. Extraction of oryzanols from rice bran soap stack with ether after acidification of soap stock with HCl [Tomotaro, tsuchiya et al; Jap Pat 4895 (1957)]. Isolation of oryzanols by transesterification if rice bran dark oil with methanol and sulfuric acid followed by column chromatography over pretreated Amberlite IRA-401 using mixed solvent methanol and ether as the eluent [Tomaro, Tsuchiya and Osamu, Okubo; Jap Pat 13649 (1961)]. Isolation of 98.3% pure oryzanols in overall about 35% yield by liquid-liquid extraction of hexane solution of 20.2% concentrated solution of oryzanol using water-saturated furfural as the extractant [Watanabe, Vasuo et al; Jap Pat 7812730 (1968)] and extraction of oryzanols from the raw oils of rice bran and ferment, maize and barley by distillation of these oils at comparatively low temperature followed by extracting the residue with hydrox123yl solvents [Yamamoto, Takeshi, Ger Pat 1301002 (1969)]. Process for the isolation of oryzanols from crude dark acid oil (rice bran) [Das; P. K, Chaudary; A, Kaimal; T. N. B, Bhalerao; U. T, U.S. Pat. No. 5,869,708 (1999)]. Process for the isolation of oryzanols from rice bran oil soap stock [Rao; K. V. S. A, Rao; B. V. S. K. Kaimal; T. N. B, U.S. Pat. No. 6,410,762 (2002)]. The Hypocholesterolemic activity of rice bran oil has been shown to be due to its constituent oryzanol and to some other component of the unsaponifiable matter [Seetharamaiah, G. S, and Chandrasekhara, N. Atherasclerosis: 78.219 (1989)].

The important biological activity of oryzanol as its cholesterol lowering property. Lipid peroxidation has been shown to be prevented in the retina by γ-Oryzanol because of its antioxidant property [Heramitsu, Tadahisa and Armstrong Donald, Opthalmic Res: 23, 196 (1991)]. Pharmaceutical preparation containing oryzanols have been shown to successfully reduce wrinkles in aged woman Sakai, [Tatsu et al (Eisai Co Ltd.) JP 05.30.526 (1993)]. Nail lacquers containing oryzanols prevent discoloration of nails [Ito, Nobumasa (Polo Chemical Industries Inc.), JP 02.290.806 (1990)]. Deodorant formulations containing oryzanols are especially effective in controlling odor from perspiration and underarms [Kumasaka, Sadao (Human industry Corp.) JP 633322 (1988)]. Oryzanol containing pharmaceutical formulations are used in preventing motion sickness [Sakada, Hideharu; JP 82.32.229 (1982)] and in the treatment of nervous system disorder [Sun. Zhide and Cong Yizi; CN 87,101,519 (1998)]. A plethora or oryzanol containing transdermal pharmaceutical and moisturizing cosmetic preparations have been prepared for the treatment of skin disorders [Courtin, Olivier (Clarins S. A.), FR 2,688,137 (1993); Tokuda, Yasuaki et al (Nisei Marine Kogyo K.K.). JP 01,290, 613 (1998); Ichimaru Co. Ltd. 16, JP 81,161,315 (1981); Toyo Chemical Corp. JP 82,149,212 (1982); Zenyaku Kogyo Co Ltd., 82,42,621 (1982); Nitto Electric industrial Co. Ltd., JP 59,53,415 (1984), JP 59,184,120 (1984)]. Oryzanolemulsions are used as antioxidants and preservatives for cosmetics and foods and such emulsions are also effective in preventing color changes in the products. [Orita Yuka Co. Ltd., JP 58,45,728 (1983)]. Soft capsules containing oryzanols with or without riboflavin butyrate can be used to prevent arteriosclerosis [Nishin Kogaku K.K.JP 58,103,315 (1983)]. Bath preparations containing 3-20% (by weight) oryzanols are used in treatment of atopic dermatitis and senile xeroderma [Inoe, Toshio and Nunokawa Senzo (Otsuka Pharm Co. Ltd., JP 05,279,272 (1993)]. Oryzanols have been shown to be highly effective against lipogenic liver cirrhosis in spontaneously hypertensive rats, an animal having natural abnormalism in lipid metabolism [Ito, Masahiro et al; J. Clin Biochem. Nutr. 12,193 (1992)]. Investigations directed towards the safety assessment of oryzanols clearly indicate that oryzanols possess no genotoxic and carcinogenic initiation activities [Tsushimoto Gen et al, J. Toxicol. Sci. 16, 191 (1991)]. [Tamagawa, M et al. Food. Chem. Toxicol. 30.49. (1992)].

Oryzanol is sold as an agent for body building in humans, especially athletes and animals such as horses and dogs. It is claimed to help individuals involved in weight training and aerobic exercise programs as a natural steroid alternative to help develop lean muscle mass and improve definition. The antioxidant property of oryzanol is believed to be responsible for this favourable effect. A major application for oryzanol is in cosmetics due to its action on the sebaceous glands. γ-Oryzanol is also known for its protective role in UV-light induced lipid peroxidation and is being used in the sunscreen form16ulations. Ferulic acid and its esters stimulate hair growth and prevent skin aging [Jap Pat 08,81,352 (1996)]. Topical preparations useful for application to hair and skin and the preparations containing 1 weight % oryzanol have been shown to convert gray hair into natural black [Sakai Tatsu et al (Eisai Co Ltd.) JP 05 225.037 (1993)]. The multiactive effects of ferulic acid and γ-oryzanol in cosmetics have been reviewed [Jap Pat 06,48,940 (1994)]. γ-Oryzanol was solubilised into medicinal drinks by using sucrose fatty acid ester and ethoxylated HCO [Jap Pat 05,255,037 (1993)]. The decomposition of tocopherols in edible oils could be prevented by the addition of oryzanol during the early part of heating at 180° C. [Jap Pat 82,136,509 (1982)]. Ferulic acid is useful as a raw material for medicines, agricultural chemicals, cosmetics, pigments and food additives, etc. and the same can be prepared from oryzanol [Eur Pat 503,650 (1992)]. Oryzanol may also be used for the manufacture of vanillin by saponification and oxidation of the ferulic acid obtained with nitrobenzene. Other miscellaneous applications of oryzanol include antibacterial, UV-blocking fabrics, mildew-proof polypropylene, biocidal fibers, Nylon 6, wool, cotton. Rice bran oil containing inositol and/or γ-oryzanol is claimed to be useful for improving t16he quality of cooked rice.

Rice bran oil uniquely contains γ-oryzanol among common vegetable oils. The high value compound can be isolated from the inexpensive acid oil produced during alkali refining of the oil in a cost-effective manner. Currently, most of the rice bran oil processing units are shifting towards physical refining in order to arrest the higher oil loss that occurs during chemical refining. By adopting physical refining for high FFA rice bran oil, the refiner has not only been able to arrest the neutral oil loss during chemical refining but also as been able to recover fatty acid as a valuable by-product in a purer form as compare to the acid oil obtained during alkali refining process. Soap stock will not be produced during physical refining which is potential source for isolation of oryzanol. As oryzanol has significant hypocholesteremic and several other benefits, it may be appropriate to look for an alternative compound with similar activity, so that the same can be added to vegetable oils as an additive to improve their nutritional value. In the present invention, phytosterols were used for the preparation of phytosteryl ferulate, an equivalent to some of the molecules present in oryzanol isolated from rice bran oil soap-stock. Soybean and sunflower deodorizer distillates are a good source for phytosterols and preparation of phytosteryl ferulate based on phytosterols certainly enhances the value of refining by-products like deodorizer distillate.

### OBJECTIVES OF THE INVENTION

The main objective of the present invention is to provide a new synthetic process for the preparation of phytosteryl ferulate as nutraceutical/food supplement, an equivalent to some of the molecules present in oryzanol isolated from rice bran oil soap-stock using ferulic acid and phytosterols isolated from soybean oil deodorizer distillate.

Another objective of the present invention is to provide a process for the acetylation of ferulic acid over heterogeneous catalyst namely monoammonium salt of 12-tungstophospharic acid using acetic anhydride with out pyridine at ambient temperature (25-30° C.).

Yet another objective of the present invention is to provide a process for the acetylation of ferulic acid using heterogeneous catalyst namely monoammonium salt of 12-tungstophospharic acid and acetic anhydride under microwave-assisted conditions to reduce the reaction time considerably from 3 hr to 10-20 min.

Still another objective of the present invention is evaluation of phytosteryl ferulate for hypocholesteremic activity in hamsters in comparison with that of natural oryzanol isolated from rice bran oil soap stock as nutraceutical/food supplement.

### SUMMARY OF THE INVENTION

Accordingly, the present invention provides a process for the preparation of phytosteryl ferulate of general formula 1

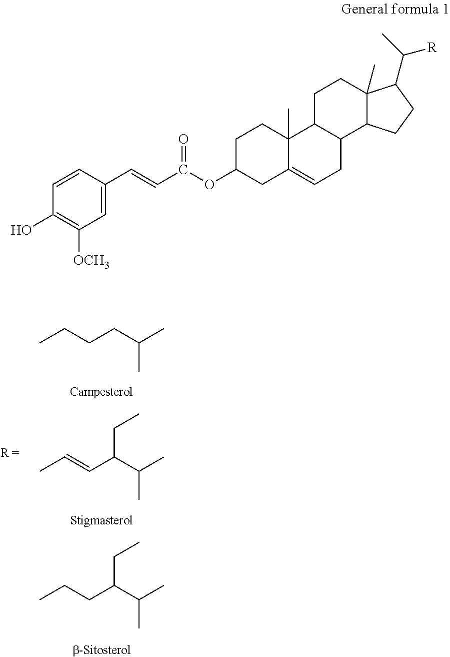

comprising the steps of: 16
(a) acetylating ferulic acid to ferulic acid acetate;
(b) esterifying ferulic acid acetate as obtained in step (a) with phytosterols by stirring at room temperature for 45-48 hr in the presence of dichloromethane, N,N′-dicyclohexyl-carbodiimide (DCC) and 4-dimethylaminopyridine (DMAP) to obtain phytosteryl ferulate acetate along with by-product dicyclohexylurea (DCU);
(c) filtering the reaction product as formed in step (b) to remove by-product dicyclohexylurea (DCU) from phytosteryl ferulate acetate;
(d) purifying phytosteryl ferulate acetate as obtained in step (c) by column chromatography;
(e) deprotecting purified phytosteryl ferulate acetate as obtained in step (d) to obtain phytosteryl ferulate;
(f) purifying phytosteryl ferulate as obtained in step (e) by column chromatography.

In one embodiment of the invention, the ferulic acid acetate was synthesized by acetylation of ferulic acid with acetic anhydride and pyridine or acetic anhydride and heterogeneous solid catalyst monoammonium salt of 12-tungstophosphoric acid [(NH₄)H₂PW₁₂O₄₀] by stirring or alternately by microwave-irradiation.

In further embodiment of the invention, the acetylation of ferulic acid using acetic anhydride and pyridine in step (a) is carried out at 85-90 degree C. for 6-,8 hr.

In another embodiment of the invention, the acetylation of ferulic acid using monoammonium salt of 12-tungstophospharic acid in step (a) is carried out at ambient temperature (25-30° C.) for 3-5 hr.

In another embodiment of the invention, the heterogeneous catalyst used in step (a) for acetylation of ferulic acid is separated from the reaction mixture by simple filtration and the catalyst was reused without any pretreatment.

In another embodiment of the invention, the acetylation of ferulic acid using acetic anhydride and heterogeneous catalyst in step (a) is also carried out under microwave-irradiated conditions at 300-800 W and 100-120° C. for 10-20 min.

In another embodiment of the invention, the heterogeneous catalytic method is simple, nontoxic and the separation of the catalyst involves simple filtration and the catalyst can be reused.

In another embodiment of the invention, wherein ferulic acid acetate yielding in the range of 80-95%.

In another embodiment of the invention, wherein phytosterols used are isolated from vegetable oil deodorizer distillate, selected from the group consisting of soybean and sunflower.

In another embodiment of the invention, wherein phytosterols, isolated from soybean oil deodorizer distillate contains mainly campesterol (18-23%), stigmasterol (25-35%) and β-sitosterol (41-56%).

In another embodiment of the invention, phytosteryl ferulate acetate in step (d) is selectively eluted using a solvent system of hexane, ethyl acetate and chloroform (80:5:15 by volume) from the silica gel column.

In another embodiment of the invention, the yield of the phytosteryl ferulate acetate after column chromatography is in the range of 75-80%.

In another embodiment of the invention, wherein the phytosteryl ferulate acetate is deprotected in step (e) by reacting with 1 to 3 wt % anhydrous potassium carbonate dissolved in chloroform:methanol in the ratio of 2:1 by volume to remove the acetate group from phytosteryl ferulate acetate.

In another embodiment of the invention, the yield and purity of the phytosteryl ferulate is 85-90% and 85-100% respectively.

In yet another embodiment of the invention, the phytosteryl ferulate was purified in step (f) by silica gel column chromatography using hexane, ethyl acetate and chloroform (80:5:15 by volume) as the eluting solvents.

In still another embodiment of the invention, wherein the said phytosteryl ferulate prepared from ferulic acid and soybean phytosterols as nutraceutical/food supplement is evaluated for hypocholesteremic activity in hamsters, exhibiting hypocholesteremic activity at par in comparison with natural oryzanol isolated from rice bran oil soap-stock.

In still another embodiment of the invention, the said phytosteryl ferulate even though comprises only three compounds namely, campesteryl ferulate, stigmasteryl ferulate and β-sitosteryl ferulate exhibited similar hypocholesteremic activity as that of natural oryzanol comprising five compounds namely, cycloartanyl ferulate, cycloartenyl ferulate, 24-methylenecycloartenyl ferulate, campesteryl ferulate and sitosteryl ferulate.

### BRIEF DESCRIPTION OF THE FIGURES

For a better understanding of the invention an exemplary embodiment is described below considered together with the figures in which:

FIG. 1. Molecules Present in γ-Oryzanol Isolated from Rice Bran Oil

FIG. 2. Scheme-1: Preparation of Phytosteryl Ferulate

### DETAILED DESCRIPTION OF THE INVENTION

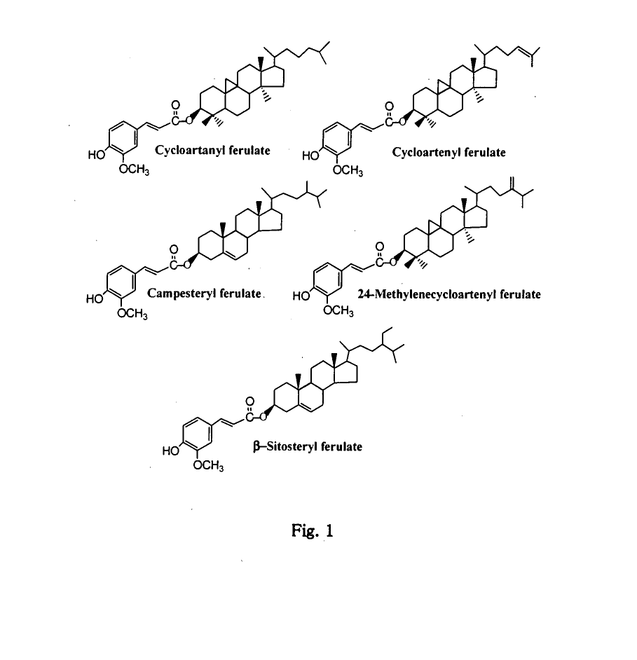

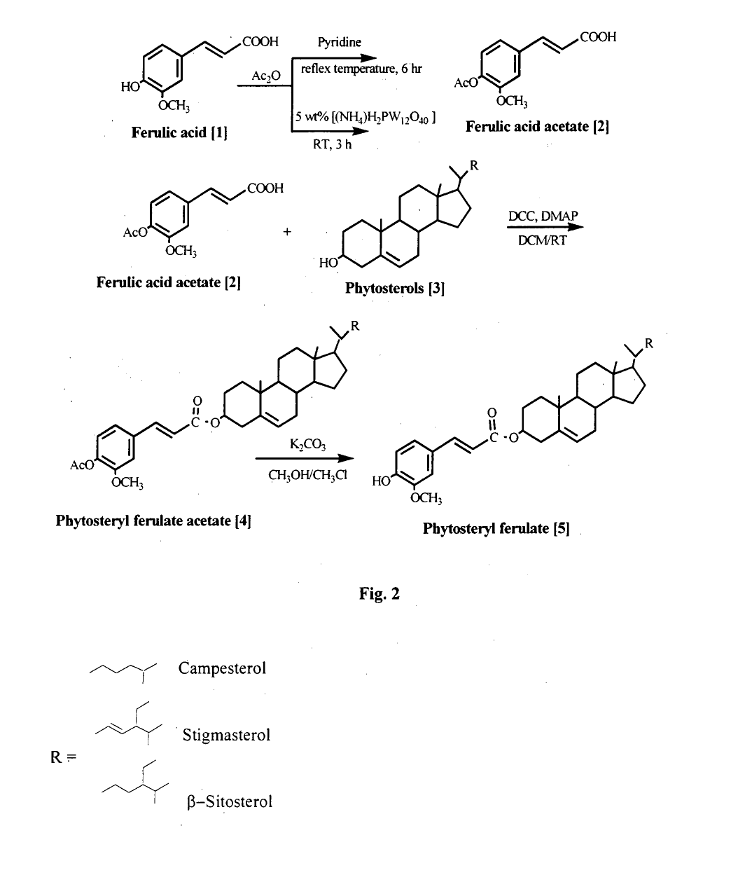

The present invention relates to a new synthetic process for the preparation of ferulic acid esters of phytosterols namely phytosteryl ferulate (Scheme 1/FIG. 2) as nutraceutical/food supplement, as an equivalent to natural oryzanol (FIG. 1) isolated from rice bran oil soap stock using ferulic acid and phytosterols (isolated from soybean oil deodorizer distillate). The soybean sterols contain mainly campesterol, stigmasterol and β-sitosterol whereas rice bran oil sterols contain in addition triterpene alcohols. In the present invention, initially, ferulic acid acetate, (2) was prepared in 80% yield by treating ferulic acid, (1) with acetic anhydride and pyridine (Scheme 1/FIG. 2) at 85-90 degree C. for 6 hr. After completion of the reaction, pyridine was quenched by aqueous HCl solution and the product was extracted with ether. This method was found to be tedious and handling pyridine also complicate. In order to avoid pyridine in the reaction an alternate simple heterogeneous catalyst was employed. The application of heterogeneous catalysts in acetylation is preferable since these can be easily separated from the reaction products by filtration and which facilitates the use of continuously operated reactors like fixed bed reactors.

Heterogeneous catalyst namely monoammonium salt of 12-tungstophosphoric acid prepared by our group [B. Y. Giri, K. Narasimha Rao, B. L. A. Prabhavathi Devi, N. Lingaiah, I. Suryanarayana, R. B. N. Prasad and P. S. Sai Prasad, Catalysis Communications, 6, (2005) p. 788-792], was employed for the preparation of ferulic acid acetate. Monoammonium salt of 12-tungstophosphoric acid was prepared by simple ion exchange of 12-tungsto phosphoric acid with a required amount of ammonium carbonate in aqueous medium. During this process, water-soluble 12-tungstophosphoric acid transforms into its insoluble monoammonium salt. After the exchange, the catalyst was calcined in air at 350° C. for 4 hr.

Ferulic acid (1) was treated with acetic anhydride in presence of 5 wt % of the heterogeneous catalyst namely monoammonium salt of 12-tungstophosphoric acid for 3 hr at room temperature (25-30° C.). The reaction was monitored by TLC and after completion of the reaction excess acetic anhydride was removed under vacuum. The reaction mixture was taken in ethyl acetate and filtered the catalyst. The solvent was removed under reduced pressure to obtain the ferulic acid acetate (2) in 92-95% yield (Scheme 1/FIG. 2).

The preparation of ferulic acid acetate has been carried out by employing monoammonium salt of 12-tungstophosphoric under microwave-irradiation to get the ferulic acid acetate in 92-95% yields within 10 min. A mixture of 4-hydroxy-3-methoxy-cinnamic acid (1), acetic anhydride and monoammonium salt of 12-tungstophosphoric acid was irradiated in microwave accelerated reaction at 100° C. for 10 min. Reaction mixture was monitored by TLC and after completion of the reaction, chloroform was added to the reaction mixture. The reaction mixture was filtered to separate the catalyst. The solvent was removed under reduced pressure. Methanol was added to dissolve the compound under heating and then cooled to 25-30 degree C. to precipitate the product. The product was filtered, dried to obtain white colored solid of 4-acetyloxy-3-methoxy-cinnamic acid (2, ferulic acid acetate) was obtained in 92% yield. The product was characterized by spectroscopic methods such as NMR, IR and GC-MS. The monoammonium salt of 12-tungstophosphoric acid catalyzed reaction gave excellent yields under microwave reaction conditions in relatively very short reaction time.

The ferulic acid acetate (2) was coupled with a mixture of phytosterols isolated from soybean oil deodorizer distillate in the presence of dicyclohexacarbodiimide (DCC) and dimethyl aminopyridine (DMAP) in dichloromethane to get phytosteryl ferulate acetate (3) in 75-80% yield. At this stage purification of the phytosteryl ferulate acetate was optimized by silica gel column chromatography using hexane, ethyl acetate and chloroform (80:5:15, v/v/v) as eluant. The pure phytosteryl ferulate acetate (3) was treated with K₂CO₃ in chloroform/methanol (2:1) mixture at reflux temperature (60-65 degree C.) to de-protect the acetate group to obtain phytosteryl ferulate (4) in 90-95% yield.

Cholesterol lowering studies of phytosteryl ferulate as nutraceutical/food supplement prepared from soybean phytosterols and ferulic acid in comparison with natural oryzanol isolated from rice bran oil soap stock were conducted. Phytosteryl ferulate (synthetic oryzanol) was significantly lowered the cholesterol, LDL cholesterol and triglycerides and increased the HDL cholesterol in both the study designs (Method A & Method B) indicating that the phytosteryl ferulate lowers the elevated cholesterol levels and also interferes with the absorption of cholesterol. The effect is comparable with that of natural oryzanol isolated from rice bran oil soap stock and phytosteryl ferulate may be used as synthetic oryzanol. The phytosteryl ferulate even though comprises only three compounds namely, campesteryl ferulate, stigmasteryl ferulate and β-sitosteryl ferulate exhibited similar hypocholesteremic activity as that of natural oryzanol comprising five compounds namely, cycloartanyl ferulate, cycloartenyl ferulate, 24-methylenecycloartenyl ferulate, campesteryl ferulate and sitosteryl ferulate.

The following examples are given by way of illustration and therefore, should not be construed to limit the scope of the present invention

### Example 1

Acetylation of 4-hydroxy-3-methoxy-cinnamic acid (ferulic acid) was carried out (Scheme 1) by treating ferulic acid (1, 10 g) with acetic anhydride (40 ml) in the presence of a pyridine (40 ml). The contents were heated at 85 degree C. for 6 hr. The reaction mixture was cooled to 30 degree C and neutralized with aqueous HCl solution and the product was extracted with ether. The ether layer was concentrated and dried under reduced pressure to get 4-acetyloxy-3-methoxy-cinnamic acid (ferulic acid acetate, 2) as powder in 80% yield. The product was characterized by spectroscopic methods such as ¹H and ¹³C NMR, IR and GC-MS.

### Spectral Data:

M.P.: 162-165° C.

IR (100: 3448, 1764, 1702, 1627, 1598 and 1512 Cm⁻¹.

¹H NMR (CDCl₃, δ): 2.3 (3H, s, OAc), 3.9 (3H, s, O—CH₃), 6.5 (1H, d, J=15.9 Hz, CH), 7.1 (1H, m Ar—H), 7.3 (2H, m, Ar—H) and 7.7 (1H, d, J=15.9 Hz, CH).

¹³C NMR (CDCl₃, δ): 20.4 (C═O), 55.9 (OCH₃), 111.7, 119.5, 121.5, 123.5, 133.3, 140.8, 143.3, 151.2, 167.6 and 168.4.

Mass (GC-MS): 236, 194, 179, 133 and 77.

### Example 2

The heterogeneous acid catalyst, monoammonium salt of 12-tungstophosphoric acid was prepared by [B. Y. Giri, K. Narasimha Rao, B. L. A. Prabhavathi Devi, N. Lingaiah, I. Suryanarayana, R. B. N. Prasad and P. S. Sai Prasad, Catalysis Communications, 6, (2005) p. 788-792] simple ion exchange of 12-tungsto phosphoric acid with a calculated amount of ammonium carbonate in aqueous medium such that one proton of the acid is replaced by one ammonium ion. After the exchange, the catalyst was calcined in air at 350° C. for 4 hr. The retention of Keggin structure was identified by XRD and FT-IR, after the ion exchange. BET surface area of the catalyst was determined in an all-glass high vacuum apparatus, adsorbing nitrogen at liquid nitrogen temperature. The X-ray diffraction pattern was obtained on a Siemens D-5000 diffractometer; using Cu K_{∝} radiation was employed for the preparation of ferulic acid acetate.

### Example 3

A mixture of 4-hydroxy-3-methoxy-cinnamic acid (1, ferulic acid, 20 g), acetic anhydride (40 ml) and monoammonium salt of 12-tungstophosphoric acid (1 g, 5 wt. %) was stirred at 30 degree C for 3 hr. Reaction mixture was monitored by TLC and after completion of the reaction, the reaction mixture was filtered to separate the catalyst. The excess acetic anhydride was removed under reduced pressure to get the solid residue. Methanol (50 ml) was added to the residue and heated to dissolve and then cooled to 25 degree C to crystallize 4-acetyloxy-3-methoxy-cinnamic acid (2, ferulic acid acetate) in 92% yield. The products were characterized by spectroscopic methods such as ¹H and ¹³C NMR, IR and GC-MS and the data obtained was same as given in example 1.

### Example 4

A mixture of 4-hydroxy-3-methoxy-cinnamic acid (5 g) (1), acetic anhydride (10 ml) and monoammonium salt of 12-tungstophosphoric acid (250 mg) was irradiated in microwave accelerated reaction at 100° C. for 10 min at 300 W using Microwave Reactor (MARS5), CEM Corporation, USA. Reaction mixture was monitored by TLC and after completion of the reaction, chloroform (25 ml) was added and filtered to separate the catalyst. The solvent was removed under reduced pressure and dissolved in hot methanol (10 ml) and crystallized the product at 30 degree C. The product 4-acetyloxy-3-methoxy-cinnamic acid (2, ferulic acid acetate) was obtained in 92% yield. The product was characterized by spectroscopic methods such as ¹H and ¹³C NMR, IR and GC-MS and the data obtained was same as given in example 1.

### Example 5

Isolation of Phytosterols from Soybean Oil Deodorizer Distillate (DOD):

Soybean oil DOD (2 kg) containing about 8% phytosterols and 6% tocopherols was enriched with phytosterols by distilling out the fatty acids at 170° C. under reduced pressure (0.01 mm) using short path distillation unit. The residue contains about 10% of free fatty acid along with 50% of triglyceride was simultaneously esterified and transesterified to fatty acid methyl esters and distilled the methyl esters using short path distillation unit at 120° C. at reduced pressure (0.01 mm). The residue contained about 26% tocopherol and 46% of phytosterols. The phytosterols were crystallized out from the residue using aqueous methanol (5%) as solvent. The composition of phytosterols mixture was found to be campesterol (19.6%), stigmasterol (27.1%) and β-sitosterol (53.4%) by HPLC analysis. This phytosterol mixture was directly used for the preparation of phytosteryl ferulate as a substitute for gamma oryzanol.

### Example 6

4-Acetyloxy-3-methoxy-cinnamic acid (2, ferulic acid acetate, 20 g) obtained in Example 3 and soybean phytosterols (3, 38.6 g) having the composition of as given in Example 5 were dissolved in dry dichloromethane (30 ml) and dicyclohexyl carbodiimide (15.0 g) and 4-dimethyl amino pyridine (1.0 g) were added. The reaction mixture was stirred at 25° C. for 48 hr. After completion of the reaction, the by-product dicyclohexyl urea was removed from the reaction mixture. The filtrate was concentrated and dried under vacuum. The crude product was purified by silica gel (100-200 mesh) column chromatography using a mixture of hexane, ethyl acetate and chloroform (80:5:15, v/v/v) as eluants to obtain pure steryl-4-acetyloxy-3-methoxy-cinnamate (4, phytosteryl ferulate acetate) in 80% yield. The product was characterized by ¹H and ¹³C NMR, IR and mass spectral analysis.

### Spectral Data:

M.P: 197-203° C.

IR (KBr): 2957, 2868, 1758, 1716, 1636, 1601, 1513, 1155 and 1031 Cm⁻¹.

¹H NMR (CDCl₃, 8): 0.65-2.05 (sterol moiety ˜48H), 2.30 (s, OAc), 3.85 (3H, s, OCH₃), 4.70 (1H, m), 5.4 (1H, t), 6.27-6.35 (1H, d, J=16.10 Hz, CH), 7.60 (1H, d, J=16.10 Hz, CH) and 7.10 (3H, m ArH).

¹³C NMR (CDCl₃, δ): 11.85, 11.98, 12.21, 18.78, 19.05, 19.32, 20.59, 21.05, 23.09, 24.29, 26.15, 27.9, 28.22, 29.2, 31.9, 33.97, 36.63, 37.03, 38.23, 39.75, 42.33, 45.87, 50.08, 55.88, 56.71, 74.18, 76.58, 77.0, 77.42, 111.2, 118.95, 121.17, 122.73, 123.2, 133.5, 139.64, 141.38, 143.64, 151.38, 166.15 and 168.68.

Mass (EI): 632, 590, 395.

### Example 7

Steryl-4-acetyloxy-3-methoxy-cinnamate (4, phytosteryl ferulate acetate, 25.0 g) obtained from soybean phytosterols as in Example 6 was dissolved in 2:1 ratio mixture of chloroform:methanol (100 ml) and potassium carbonate (2.0 g) was added and heated at 65 degree C. for 6 hr. The reaction mixture was neutralized with 50 ml of aqueous ammonium chloride solution and extracted with chloroform (3×150 ml). The organic layer was concentrated and dried under reduced pressure to get the residue and was purified by column chromatography to obtain the pure phytosteryl ferulate (5) in 95-97% yield. The product was characterized by ¹H and ¹³C NMR, IR and mass spectral analysis. The phytosteryl ferulate composition was determined by HPLC and found to be campesteryl ferulate (18.9%), stigmasteryl ferulate (29.1%) and P-sitosteryl ferulate (52.0%).

### Spectral Data:

M.P.: 196-204 C.

IR (KBr): 3421, 2954, 2868, 1705, 1633, 1598, 1515, 1268, 1165 and 1029 Cm⁻¹.

¹H NMR (CDCl₃, δ): 0.7-2.0 (β-sitosterol moiety ˜48H), 3.95 (31-1, s, O—CH₃), 4.73 (1H, m), 5.4 (1H, t), 6.22 (1H, d, J=15.90 Hz, CH), 6.95-7.10 (3H, m ArH) and 7.60 (1H, d, J=15.90, Hz, CH).

¹³C NMR (CDCl₃, 8): 11.85, 11.98, 12.21, 18.78, 19.05, 19.33, 21.04, 23.09, 24.29, 26.16, 27.9, 28.22, 29.2, 31.9, 33.97, 36.63, 37.05, 38.28, 39.75, 42.33, 45.87, 8 50.08, 55.91, 56.71, 73.92, 76.57, 77.0, 77.42, 109.33, 114.72, 116.1, 122.65, 122.99, 127.12, 129.31, 139.72, 144.47, 146.77, 147.89 and 166.63.

Mass (EI): 590 (M+), 396, 193.

### Example 8

Syrian Male Hamsters of body weights in the range of 90 g to 110 g were used as test animal in the present study. They were divided into 3 groups, each consisting of 6 animals. The basal diet was in the form of purified diet according to AlN 93 recommendations, (ref) which was fortified with 1% cholesterol and 10% coconut oil (henceforth called high cholesterol diet, HCD in this invention). Diet for the test groups was prepared by mixing of 1% each of natural oryzanol and phytosteryl ferulate (synthetic oryzanol) with the control group's diet, HCD. Each hamster was kept in a cage at a constant temperature of 22±1° C. and a relative humidity of 55±5% during the entire feeding regime. All the animals in all the groups were fed with HCD for a period of 5 weeks till the induction of hypercholesteremia is achieved. At this stage, hamsters in the two treated groups were fed with HCD containing 1.0% of natural oryzanol isolated from rice bran oil soap stock and 1.0% of phytosteryl ferulate prepared from ferulic acid and soybean phytosterols (synthetic oryzanol) respectively, whereas the control group received the HCD alone. The feeding continued further for a period of 11 weeks. On 77^{th} day, hamsters were fasted overnight and blood was taken from each hamster through the descending abdominal arota under anesthesia. The total lipid profile of each hamster, such as total cholesterol (TC), LDL (Low density lipoprotein)-cholesterol (LDL-C), HDL (High density lipoprotein)-cholesterol (HDL-C) and triacylglycerol (TG) in the serum were measured using automatic, blood analyzer, Express Plus, Bayer Diagnostics, USA. Body weights of all the animals were determined on weekly interval basis. General cage observations were also assessed every day.

The objective of this hypocholesteremic activity test was to evaluate the effect of phytosteryl ferulate prepared from ferulic acid and soybean phytosterols (synthetic oryzanol) and natural oryzanol isolated from rice bran oil soap stock in lowering the elevated cholesterol level of hypocholesteremic hamsters as nutraceutical/food supplement. Results of the hypocholesteremic activity test, according to Method A are shown in Tables 1 and 2. There is significant reduction of LDL cholesterol (p<0.01) in both the treated groups containing phytosteryl ferulate (synthetic oryzanol) and natural oryzanol isolated from rice bran oil soap stock in the HCD compared to control group, fed on HCD. Similar result was obtained in case of TG (p<0.05). The reduction in the level of TC in the treated group is quite significant (p<0.05) compared to control group. These results indicate that the phytosteryl ferulate (synthetic oryzanol) is comparable in activity with that of the natural oryzanol isolated from rice bran oil soap stock in lowering the LDL cholesterol. However, there is marginal increase in the level of HDL-C in the treated group compared to control group. Almost identical observation was observed in all these parameters in both the treated group, which indicates that the hypocholesteremic activity of the phytosteryl ferulate (synthetic oryzanol) is comparable with that of the natural oryzanol as nutraceutical/food supplement. Similar trends were observed when the percent reduction of the LDL-C, TC and TG in the treated groups was compared. The percent reduction of the LDL-C (50.2%), TC (22.1%) and TG (25.2%) in the treated group, fed on diet containing phytosteryl ferulate is comparable with those obtained in the other treated group, fed on diet containing natural oryzanol (55.9%, 23.6% and 31.2% respectively for LDL-C, TC and TG). These results indicate that the hypocholesteremic activity of the phytosteryl ferulate is comparable with the natural oryzanol. Increase in mean body weights of hamsters taken for hypocholesteremic activity test in all the groups in Method A are shown in Table 3. Identical gain in body weights was observed in all the studied groups.

TABLE 1

| | | | | | HDL-C | | | |
|---|---|---|---|---|---|---|---|---|
| | TC | | LDL-C | | Mean | | TG | |
| | Mean | | Mean | | (mg/ | | Mean | |
| Sample | (mg/dl) | SD | (mg/dl) | SD | dl) | SD | (mg/dl) | SD |
| Control | 437.8 | ±48.8 | 176.8 | ±44.8 | 170.4 | ±25 | 452.8 | ±28.3 |
| 1% NO | 334.5* | ±53.6 | 77.8** | ±33.7 | 194.3 | ±39.1 | 311.7* | ±62.7 |
| 1% SO | 340.8* | ±13.8 | 101.6** | ±11.5 | 180.5 | ±18.5 | 338.7* | ±66.1 |

NO: Natural oryzanol;
SO: Phytosteryl ferulate (Synthetic oryzanol)
*Significance level (p < 0.05);
**Significance level (p < 0.01)

TABLE 2

| | % Reduction^{a} | | | |
|---|---|---|---|---|
| Sample | TC | LDL-C | HDL-C | TG |
| 1% NO | −23.6 | −55.9 | 14.1 | −31.2 |
| 1% SO | −22.1 | −50.2 | 5.9 | −25.2 |

^{a}“% of Reduction” means (L − L_{c}) × 100/Lc, where L is the concentration of lipid component at the end of study in the experimental group and L_{c} is the same in the control group

TABLE 3

| | Body weight during feed period (g ± SD) | | | | | |
|---|---|---|---|---|---|---|
| Sample | 0^{th} day | 21^{st} day | 35^{th} day | 49^{th} day | 63^{rd} day | 77^{th} day |
| Control | 101.3 ± 14.0 | 113.8 ± 14.4 | 123.7 ± 13.3 | 130.3 ± 11.0 | 132.0 ± 9.1 | 136.2 ± 9.1 |
| 1% NO | 98.3 ± 12.5 | 113.5 ± 15.3 | 124.3 ± 16.4 | 125.8 ± 20.2 | 128.0 ± 19.8 | 132.8 ± 23.3 |
| 1% SO | 95.3 ± 16.8 | 110.7 ± 18.1 | 118.5 ± 16.2 | 122.5 ± 17.3 | 125.8 ± 17.4 | 134.2 ± 17.5 |

### Example 9

Syrian Male Hamsters of body weights in the range of 90 g to 110 g were used as test animal in the present study. They were divided into 3 groups, each consisting of 6 animals. Each hamster was kept in a cage at a constant temperature of 22±1° C. and a relative humidity of 55±5% during the entire feeding regime. The control group was fed on HCD diet, whereas the two treated groups were fed on HCD containing 1% natural oryzanol isolated from rice bran oil soap stock and phytosteryl ferulae prepared from ferulic acid and soybean phytosterols (synthetic oryzanol) respectively. The feeding was continued for a period of 11 weeks. On 77^{th} day, hamsters were fasted overnight (16 hrs) and blood was taken from each hamster through the descending abdominal arota under anesthesia. The total lipid profile of each hamster, such as total cholesterol (TC), LDL-cholesterol (LDL-C), HDL-cholesterol (HDL-C) and triacylglycerol (TG) in the serum were measured using automatic blood analyzer, Express Plus, Bayer Diagnostics, USA. Body weights of all the animals were determined on weekly interval basis. General cage observations were also assessed every day.

The objective of this hypocholesteremic activity test was to evaluate the effect of natural oryzanol and phytosteryl ferulate prepared from ferulic acid and soybean phytosterols on the rate of inhibition of absorption of cholesterol. Results of the hypocholesteremic activity test, according to Method B are shown in Tables 4 and 5. There is significant reduction of LDL cholesterol (p<0.001) in both the treated groups containing phytosteryl ferulate prepared from ferulic acid and soybean phytosterols (synthetic oryzanol) and natural oryzanol isolated from rice bran oil soap stock in the HCD compared to control group, fed on HCD. However, appreciable changes in the lipid profile were observed from the 6^{th} week onwards in both the treated groups. The initial lag phase may be due to the fact that the hamsters are normal and not hypercholesteremic in this design. Hence, the effect of natural oryzanol or phytosteryl ferulate (synthetic oryzanol) is not as pronounced as in Method A from the beginning. However, the effect of both natural oryzanol and phytosteryl ferulate (synthetic oryzanol) was seen clearly after the lag period of 6 weeks. Though somewhat decreasing trend in the level of TC and TG was observed in the treated groups, the decrease is not significant enough (p>0.05) in case of phytosteryl ferulate (synthetic oryzanol). There is significant increase in the level of HDL-C in both the treated groups (p<0.05) compared to control group. The percent reduction of the LDL-C (25.6%). TC (22.7%) and TG (15.1%) in the treated group, fed on diet containing phytosteryl ferulate is comparable with those obtained in the other treated group, fed on diet containing natural oryzanol (26.8%, 24.7% and 18.7% respectively for LDL-C, TC and TG). Even the percent increase in the level of HDL-C in these treated groups is quite comparable too (25.6% vs. 26.8%). These results indicate that the hypocholesteremic activity of the phytosteryl ferulate (synthetic oryzanol) is comparable with that of the natural oryzanol. Increase in mean body weights of hamsters taken for hypocholesteremic activity test in all the groups in Method B are shown in Table 3. Identical gain in body weights was observed in all the studied groups.

TABLE 4

| | TC | | | | HDL-C | | | |
|---|---|---|---|---|---|---|---|---|
| | Mean | | LDL-C | | Mean | | TG | |
| Sample | (mg/dl) | SD | Mean (mg/dl) | SD | (mg/dl) | SD | Mean (mg/dl) | SD |
| Control | 491.0 | ±69.5 | 240.5 | ±35.2 | 144.3 | ±22.8 | 601.5 | ±69.5 |
| 1% NO | 369.5* | ±18.6 | 88.7*** | ±12.1 | 183* | ±22.2 | 488.8* | ±59.5 |
| 1% SO | 379.7 | ±49.2 | 104.2*** | ±10.4 | 181.3* | ±17.5 | 510.0 | ±52.3 |

NO: Natural oryzanol;
SO: Phytosteryl ferulate (Synthetic oryzanol)
*Significance level (p < 0.05);
***Significance level (p < 0.001)

TABLE 5

| | % Reduction^{a} | | | |
|---|---|---|---|---|
| Sample | TC | LDL-C | HDL-C | TG |
| 1% NO | −24.7 | −63.1 | 26.8 | −18.7 |
| 1% SO | −22.7 | −56.6 | 25.6 | −15.1 |

^{a}“% of Reduction” means (L − L_{c}) × 100/L_{c,} where L is the concentration of lipid component at the end of study in the experimental group and L_{c} is the same in the control group

TABLE 6

| | Body weight during feed period (g ± SD) | | | | | |
|---|---|---|---|---|---|---|
| Sample | 0^{th} day | 21^{st} day | 35^{th} day | 49^{th} day | 63^{rd} day | 77^{th} day |
| Control | 94.4 ± 23.4 | 111.6 ± 24.3 | 113.2 ± 24.4 | 117.2 ± 22.1 | 116.4 ± 17.7 | 118.4 ± 11.8 |
| 1% NO | 103.2 ± 10.3 | 114.3 ± 12.2 | 116.2 ± 7.6 | 114.8 ± 8.2 | 115 ± 6.5 | 115.5 ± 7.4 |
| 1% SO | 107.3 ± 13.6 | 111.3 ± 17.4 | 121.3 ± 18.2 | 122.5 ± 17.6 | 119.7 ± 15.4 | 120.2 ± 16.4 |

In total, phytosteryl ferulate prepared from ferulic acid and soybean phytosterols (synthetic oryzanol) has significantly lowered the cholesterol, LDL cholesterol and triglycerides and increased the HDL cholesterol in both the study designs indicating that the phytosteryl ferulate prepared from ferulic acid and soybean phytosterols (synthetic oryzanol) lowers the elevated cholesterol levels (Therapeutic action) and also interferes with the absorption of cholesterol (Prophylactic action). The effect is comparable with that of natural oryzanol isolated from rice bran oil soap stock as nutraceutical/food supplement.

### ADVANTAGES OF THE INVENTION

1. The present invention provide a new synthetic process for the preparation of phytosteryl ferulate as nutraceutical/food supplement, an equivalent to some of the molecules present in oryzanol isolated from rice bran oil soap-stock using ferulic acid and phytosterols isolated from soybean oil deodorizer distillate.
2. phytosteryl ferulate as prepared in present invention shows hypocholesteremic activity in hamsters in comparison with that of natural oryzanol isolated from rice bran oil soap stock at par.

We claim:

1. A process for the preparation of phytosteryl ferulate of general formula 1 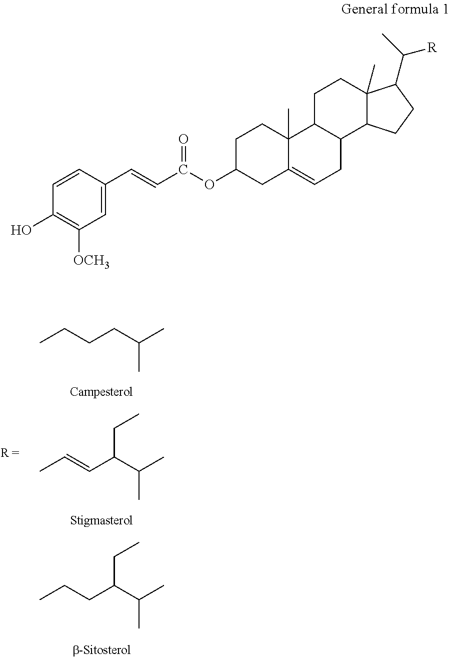
comprising the steps of:
a. acetylating ferulic acid to ferulic acid acetate;
b. esterifying ferulic acid acetate as obtained in step (a) with phytosterols by stirring at room temperature for 45-48 hr in the presence of dichloromethane, N,N′-dicyclohexyl-carbodiimide (DCC) and 4-dimethylaminopyridine (DMAP) to obtain phytosteryl ferulate acetate along with by-product dicyclohexylurea (DCL);
c. filtering the reaction product as formed in step (b) to remove by-product dicyclohexylurea (DCU) from phytosteryl ferulate acetate;
d. purifying phytosteryl ferulate acetate as obtained in step (c) by column chromatography;
e. deprotecting purified phytosteryl ferulate acetate as obtained in step (d) to obtain phytosteryl ferulate;
f. purifying phytosteryl ferulate as obtained in step (e) by column chromatography.

2. A process as claimed in claim 1, wherein the ferulic acid acetate in step (a) was synthesized by acetylation of ferulic acid with (i) acetic anhydride and pyridine; or (ii) acetylation of ferulic acid with acetic anhydride and heterogeneous solid catalyst monoammonium salt of 12-tungstophosphoric acid [(NH₄)H₂PW₁₂O₄₀] by stirring; or (iii) by acetylating with acetic anhydride and heterogeneous solid catalyst monoammonium salt of 12-tungstophosphoric acid [(NH₄)H₂PW₁₂O₄₀] by microwave-irradiation.

3. A process as claimed in claim 2, wherein the acetylation of ferulic acid using acetic anhydride and pyridine in step (a) is carried out at 85-90° C. of acetic anhydride for a period ranging 6-8 hr.

4. A process as claimed in claim 2, wherein, the acetylation of ferulic acid using monoammonium salt of 12-tungstophospharic acid in step (a) is carried out at ambient temperature (25-30° C.) for 3-5 hr.

5. A process as claimed in claim 2, wherein the heterogeneous catalyst used in step (a) for acetylation of ferulic acid is separated from the reaction mixture by simple filtration and the catalyst is reused without any pretreatment.

6. A process as claimed in claim 2, wherein, the acetylation of ferulic acid using acetic anhydride and heterogeneous catalyst in step (a) is carried out under microwave-irradiated conditions at 300-800 W and 100-120° C. for 10-20 min.

7. A process as claimed in claims 3, 4 and 6, wherein yield of ferulic acid acetate obtained is in the range of 80-95%.

8. A process as claimed in claim 1, wherein phytosterols used in step (b) are isolated from vegetable oil deodorizer distillate, selected from the group consisting of soybean and sunflower.

9. A process as claimed in claim 1, wherein phytosterols, isolated from soybean oil deodorizer distillate contain mainly campesterol (18-23%), stigmasterol (25-35%) and β-sitosterol (41-56%).

10. A process as claimed in claim 1, wherein phytosteryl ferulate acetate in step (d) is selectively eluted using a solvent system of hexane, ethyl acetate and chloroform (80:5:15 by volume) from the silica gel column.

11. A process as claimed in claim 1, wherein the yield of the phytosteryl ferulate acetate after column chromatography is in the range of 75-80%.

12. A process as claimed in claim 1, wherein the phytosteryl ferulate acetate is deprotected in step (e) by reacting with 1 to 3 wt % anhydrous potassium carbonate dissolved in chloroform:methanol in the ratio of 2:1 by volume to remove the acetate, group from phytosteryl ferulate acetate.

13. A process as claimed in claim 1, wherein the phytosteryl ferulate is purified by silica gel column chromatography in step (f) using hexane, ethyl acetate and chloroform (80:5:15 by volume) as the eluting solvents.

14. A process as claimed in claim 1, wherein the yield and purity of the phytosteryl ferulate in step (f) is in the range 85-90% and 85-100% respectively.

15. A process as claimed in claim 1, wherein phytosteryl ferulate prepared and evaluated for hypocholesteremic activity in hamsters, exhibited hypocholesteremic activity at par in comparison with natural oryzanol isolated from rice bran oil soap-stock.

16. A process as claimed in claim 1, wherein the said phytosteryl ferulate even though comprises only three compounds namely, campesteryl ferulate, stigmasteryl ferulate and β-sitosteryl ferulate exhibited similar hypocholesteremic activity as that of natural oryzanol comprising five compounds namely, cycloartanyl ferulate, cycloartenyl ferulate, 24-methylenecycloartenyl ferulate, campesteryl ferulate and sitosteryl ferulate.
